(19) United States
(12) Patent Application Publication
(10) Pub. No.: US 2015/0094723 A1
(43) Pub. Date: Apr. 2, 2015

(54) ULTRASONIC SURGICAL APPARATUS WITH POWER REDUCTION

(71) Applicant: MISONIX INCORPORATED, FARMINGDALE, NY (US)

(72) Inventor: Alexander DARIAN, Brightwaters, NY (US)

(73) Assignee: MISONIX INCORPORATED, FARMINGDALE, NY (US)

(21) Appl. No.: 14/041,605

(22) Filed: Sep. 30, 2013

(51) Int. Cl. A61B 17/32; A61B 17/16

(52) U.S. Cl. CPC A61B 17/320068; A61B 17/16; A61B 2017/00039
USPC 606/79

(57) ABSTRACT

An ultrasonic surgical includes a probe with an operative edge or surface at a distal end, an electromechanical transducer connected to the probe, and an activation circuit connected to the transducer for supplying thereto an electrical waveform having an ultrasonic frequency and an amplitude. The activation circuit includes a signal generator and further includes a power reduction circuit operatively connected to the signal generator for inducing the signal generator to automatically reduce the amplitude of the waveform upon a sensing by the power reduction circuit of reduced load on the probe.

### BACKGROUND OF THE INVENTION

This invention relates to an ultrasonic surgical apparatus. More particularly, this invention relates to an ultrasonic bone cutting apparatus. The invention also relates to an associated surgical method.

Ultrasonic surgical blades for cutting bone have known for several years. U.S. Pat. No. 6,379,371 and U.S. Pat. No. 6,443,969 disclose such a surgical blade. Ultrasonic bone cutting tools exhibit significant advantages over more traditional instruments such as power saws and rotary cutters. Ultrasonic bone cutting blades have been devised for enabling a separation of bone and soft tissue, along an interface between the two, with a minimum of damage to the soft tissue. Such an instrument is disclosed in U.S. Pat. No. 8,343,178.

### OBJECTS OF THE INVENTION

It is an object of the present invention to provide and improved ultrasonic bone cutting apparatus.

A more particular object of the present invention is to provide an ultrasonic bone cutting apparatus the use of which entails a reduced probability of damage to soft tissues adjacent to a bone being cut.

Another particular object of the present invention is to provide an ultrasonic bone cutting apparatus configured to automatically protect soft tissues adjacent to a bone from being cut during an operation on the bone.

These and other objects of the present invention will be apparent from the descriptions and drawings herein. Although every object of the invention is attainable by at least one embodiment of the invention, there is not necessarily any single embodiment that achieves all of the objects of the invention.

### SUMMARY OF THE INVENTION

An ultrasonic surgical apparatus in accordance with the present invention comprises a probe with an operative edge or surface at a distal end, an electromechanical transducer connected to the probe, and an activation circuit connected to the transducer for supplying thereto an electrical waveform having an ultrasonic frequency and an amplitude. The activation circuit includes a signal generator and further includes a power reduction circuit operatively connected to the signal generator for inducing the signal generator to automatically reduce the amplitude of the waveform upon a sensing by the power reduction circuit of reduced load on the probe.

The power reduction circuit may include a load detector, and the load detector may include a current sensor. The load detector may further include a comparator operatively connected to the current sensor for comparing an output of the sensor with a pre-established reference value. The comparator may be a digital device, so that a digitizer is required to convert an analog output signal of the current sensor to a digital or binary signal for use by the comparator.

Pursuant to another feature of the present invention, the activation circuit includes a manually actuatable power switch and the power reduction circuit further includes a switching circuit operatively connected at inputs to the power switch and the comparator and at an output to the signal generator. The switching circuit acts to automatically override an instruction from the manual power switch in the event that a current reduction indicates that the ultrasonic blade has penetrated through a bone and encountered soft tissue adjacent thereto.

A surgical method in accordance with the present invention comprises operating an ultrasonic cutting tool to cut through bone of a patient, operating a sensor to automatically monitor load on the cutting tool during the operating of the cutting tool, automatically detecting a change in output of the sensor indicative of a reduced load on the cutting tool owing to a passing of the cutting tool at least partially through the bone, and automatically reducing power to the cutting tool upon the detecting of reduced load on the cutting tool.

The detecting of a change in output of the sensor may include comparing output of the sensor with a preselected reference value.

The reducing of power to the cutting tool may include reducing amplitude of an ultrasonic electrical waveform fed to an electromechanical transducer.

The operating of the sensor may include detecting current flow to an electromechanical transducer connected to the tool.

### BRIEF DESCRIPTION OF THE DRAWINGS

FIG. 1 is partially a block diagram and partially a schematic side elevation view, on an enlarged scale, of an ultrasonic bone cutting apparatus in accordance with the present invention.

FIG. 2 is a block diagram showing details of the apparatus of FIG. 1.

FIG. 3 is a block diagram showing further detailed structure of the apparatus of FIGS. 1 and 2.

### DETAILED DESCRIPTION

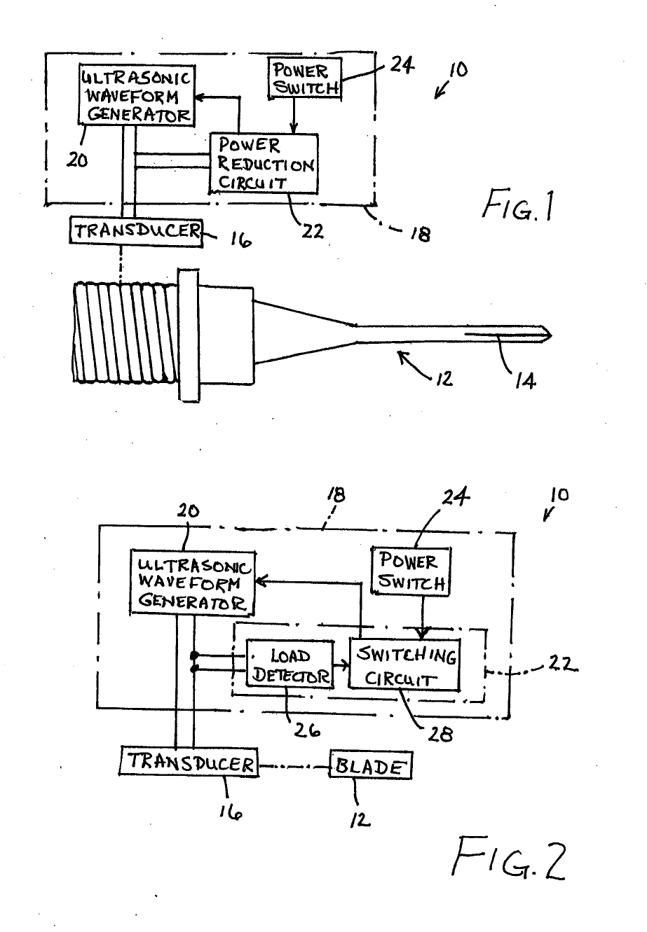

As depicted in FIG. 1, an ultrasonic surgical apparatus 10 comprises a probe or tool 12 with an operative (cutting) edge or surface 14 at a distal end. Probe of tool 12 particularly may take the form of an ultrasonic bone-cutting blade or drill. Apparatus 10 further comprises an electromechanical transducer 16 connected to probe 12 for energizing same with an ultrasonic standing wave of a preselected frequency (or wavelength) determined in part by the dimensions of the probe. Apparatus 10 also comprises an activation circuit 18 connected to transducer 16 for supplying thereto an electrical waveform having the preselected ultrasonic frequency and a predetermined amplitude. Activation circuit 18 includes a signal or waveform generator 20 and further includes a power reduction circuit 22 operatively connected to the signal generator for inducing the signal generator to automatically reduce the amplitude of the electrical waveform upon a sensing by the power reduction circuit of reduced load on probe 12.

Surgical apparatus 10 typically includes further electrical and liquid-supply components as disclosed in U.S. Pat. No. 6,379,371 and U.S. Pat. No. 6,443,969, the disclosures of which are hereby incorporated by reference. For instance, apparatus 10 may incorporate a power switch 24 such as a foot-operated switch.

As illustrated in FIG. 2, power reduction circuit 22 may include a load detector 26 and a switching circuit 28. Switching circuit 28 is connected to power switch 24 and signal generator 20 for enabling the operation of the signal generator in response to a signal from the power switch. Upon receiving a signal from load detector 26 occasioned by a drop in transducer load, switching circuit 28 induces signal generator 20 to reduce, or possibly interrupt, the power of the electrical waveform supplied to transducer 16.

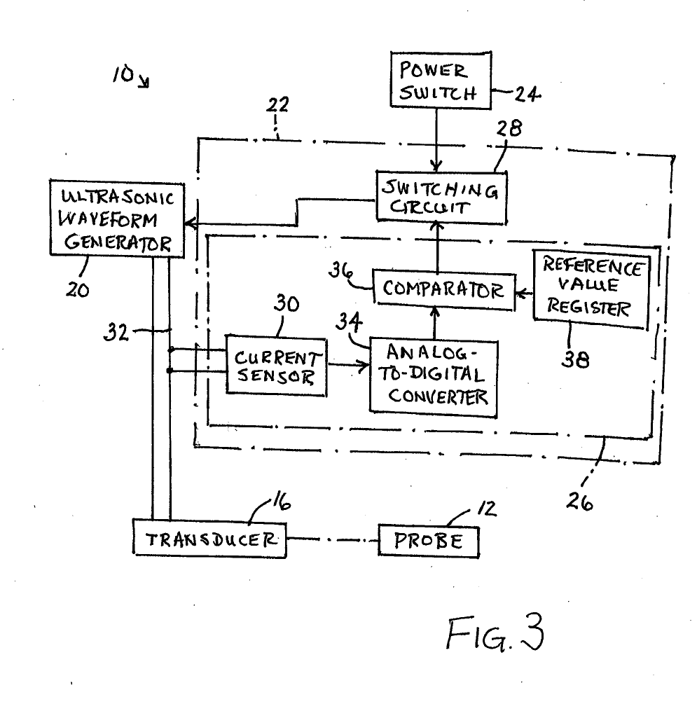

As illustrated in FIG. 3, load detector 26 may include a current sensor 30 operatively connected to a lead 32 extending from signal generator 20 to transducer 16. Load detector 26 further includes a digitizer or analog-to-digital converter 34 at an output of the current sensor 30. A comparator 36 is operatively connected to current sensor 30 via digitizer 34 for comparing an output of the sensor with a pre-established reference value provided by a register 38. Comparator 36 is thus a digital device, although an analog device such as an operational amplifier could alternatively be used.

Comparator 36 compares a digitized output of sensor 30 with the reference value from register 38 and, upon detecting that the current has fallen below a pre-established level encoded by the reference value, transmits a signal to switching circuit 28 which in turn causes signal generator 20 to reduce the power output to transducer 16, particularly by reducing the amplitude of the electrical transducer-energizing waveform. Switching circuit 28 acts to automatically override an operator-induced instruction from manual power switch 24 in the event of a current reduction resulting when ultrasonic blade or tool 12 penetrate through bone and encounters soft tissue adjacent thereto.

A surgical method utilizing the apparatus of FIGS. 1-3 entails using apparatus 10 to have ultrasonic cutting tool 12 cut through a bone of a patient. Current sensor 30 is operated to automatically monitor load on cutting tool 12 during the operation. A change in output of sensor 30 indicative of a reduced load on cutting tool 12 is automatically detected owing, the reduction in load arising from a passing of the cutting tool at least partially through the bone.

One automatically reduces power to the cutting tool 12 upon detecting reduced load on the cutting tool. The detecting of a change in output of sensor 30 may include operating comparator 36 to automatically compare output of sensor 30 with the reference value from register 38. The reducing of power to cutting tool 12 may be implemented by reducing amplitude of an ultrasonic electrical waveform fed to an electromechanical transducer, i.e., transducer 16.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

What is claimed is:

1. An ultrasonic surgical apparatus comprising: a probe including an operative edge or surface at a distal end; an electromechanical transducer connected to said probe; and an activation circuit operatively connected to said transducer for supplying thereto an electrical waveform having an ultrasonic frequency and an amplitude, said activation circuit including a signal generator and further including a power reduction circuit operatively connected to said signal generator for inducing said signal generator to automatically reduce the amplitude of said waveform upon a sensing by said power reduction circuit of reduced load on said probe.

2. The apparatus defined in claim 1 wherein said power reduction circuit includes a load detector.

3. The apparatus defined in claim 2 wherein said load detector includes a current sensor.

4. The apparatus defined in claim 3 wherein said load detector further includes a comparator operatively connected to said current sensor for comparing an output of said sensor with a pre-established reference value.

5. The apparatus defined in claim 4 wherein said activation circuit includes a manually actuatable power switch and wherein said power reduction circuit further includes a switching circuit operatively connected at inputs to said power switch and said comparator and at an output to said signal generator.

6. The apparatus defined in claim 1 wherein said power reduction circuit includes a current sensor, said sensor being operatively connected to a lead extending from said signal generator to said transducer.

7. The apparatus defined in claim 1 wherein said load power reduction circuit includes a comparator configured to compare a sensed load parameter with a pre-established reference value.

8. The apparatus defined in claim 1 wherein said activation circuit includes a manually actuatable power switch and wherein said power reduction circuit further includes a switching circuit operatively connected at an input to said power switch and at an output to said signal generator.

9. A surgical method comprising: operating an ultrasonic cutting tool to cut through bone of a patient; during the operating of said cutting tool, operating a sensor to automatically monitor load on said cutting tool; during the operating of said cutting tool, automatically detecting a change in output of said sensor indicative of a reduced load on said cutting tool owing to a passing of said cutting tool at least partially through the bone; and automatically reducing power to said cutting tool upon the detecting of reduced load on said cutting tool.

10. The method defined in claim 9 wherein the detecting of a change in output of said sensor includes comparing output of said sensor with a preselected reference value.

11. The method defined in claim 9 wherein the reducing of power to said cutting tool includes reducing amplitude of an ultrasonic electrical waveform fed to an electromechanical transducer.

12. The method defined in claim 9 wherein the operating of said sensor includes detecting current flow to an electromechanical transducer connected to said tool.
